(19) United States
(12) Patent Application Publication
(10) Pub. No.: US 2010/0185298 A1
(43) Pub. Date: Jul. 22, 2010

(54) Acetabular Spacer for Treatment of Hip Sepsis

(76) Inventor: Kenneth D. Stone, Marshfield, WI (US)

Correspondence Address: BOYLE FREDRICKSON S.C., 840 North Plankinton Avenue, MILWAUKEE, WI, 53203, US

(21) Appl. No.: 12/692,355

(22) Filed: Jan. 22, 2010

Related U.S. Application Data
(60) Provisional application No. 61/146,539, filed on Jan. 22, 2009.

(51) Int. Cl. A61F 2/34

(52) U.S. Cl. 623/22.21

(57) ABSTRACT

An acetabular spacer for maintaining hip joint position fits into the acetabulum upon removal of an acetabular implant for treatment of infection. The spacer may be composed of a bone cement material infused with antibiotic for improved insertion, treatment, and removal.

### CROSS-REFERENCE TO RELATED APPLICATIONS

This application claims the benefit of U.S. provisional application 61/146,539 filed Jan. 22, 2009 and hereby incorporated by reference.

### STATEMENT REGARDING FEDERALLY SPONSORED RESEARCH OR DEVELOPMENT

### BACKGROUND OF THE INVENTION

A natural hip may degenerate to a degree that it is necessary to replace the natural hip joint with a prosthetic hip joint. In such replacements, the head of the femur or the acetabular socket or both may be replaced with prostheses made out of biocompatible materials such as colbalt-chrome alloy, ultra high molecular weight polyethylene, or ceramic.

On occasion, the femur may become infected after the implantation of the shaft of the prosthetic femur head into the femur and the prosthetic femur head must be removed. One accepted and often preferred method of treatment is a staged replantation of the device. This is done by removing the infected femur prosthesis and replacing it with a temporary femur prosthesis that is roughly the same dimensions as the permanent femur prosthesis but loaded with antibiotics (usually PMMA cement). This temporary femur prosthesis can be more easily removed. After the temporary spacer has been in place for a period of time (usually a few weeks) a new permanent femur prosthesis is inserted back into the femur.

A temporary femur prosthesis of this type is commercially available from Exactech Inc. of Gainesville, Fla. under the trade name InterSpace™. As noted, this temporary femur prosthesis conforms generally to the shape and size of the permanent femur prosthesis and it is constructed of a biocompatible bone cement such as PMMA molded over a metallic reinforcing core and infused with an antibiotic such as Gentamicin. A ball-head of the temporary spacer may fit into the acetabulum to rest correctly against the bone of the acetabulum. The dimensions of this temporary spacer may be selected to preserve spacing between the leg and pelvis to facilitate the reinstallation of the permanent prostheses after the infection has been corrected.

Alternatively, a high density polyethylene bearing, being a portion of a commercially available acetabular prosthesis for permanent implantation, may be used as a spacer between the ball of the temporary spacer and the acetabulum to provide additional separation between the leg and pelvis during the healing process.

### BRIEF SUMMARY OF THE INVENTION

The present inventor has recognized that superior outcomes may be obtained by providing a temporary acetabular prosthesis for the acetabulum that more closely conforms to the ultimate permanent acetabular prosthesis. The difficulties of removing such a temporary acetabular prosthesis are minimized by constructing the spacer of bone cement in a pre-molded configuration providing a smooth socket and a surface that may readily adhere to additional bone cement used to install it in the acetabulum. Conventional techniques for removing bone cement may then be applied to the entire spacer. The lower wear characteristics of such a temporary acetabular spacer are acceptable in the context of its temporary nature.

Specifically then, the invention provides an apparatus for and a method of treating infection of an acetabular prosthesis. The method includes steps of removing an existing acetabular prosthesis from an acetabulum and installing a spacer in the acetabulum formed of a preformed frangible bone cement, cemented with bone cement into the acetabulum, the spacer providing a substantially semi-spherical polished socket for receiving a ball of a femur prosthesis, the spacer having substantially the dimension of the removed acetabular prosthesis. After a period of healing, the spacer is removed and a new acetabular prosthesis is installed.

It is thus a feature of at least one embodiment of the invention to provide a spacer for the acetabulum that promotes healing in the event of the need for a replant.

The polished socket may be no less than 40 mm in diameter.

It is thus a feature of at least one embodiment of the invention to provide a spacer that may work with standard femur balls, whether bone or prosthetic.

The spacer may be infused with antibiotic.

It is thus a feature of at least one embodiment of the invention to promote the healing of infected tissue.

The spacer is constructed of bone cement, for example PMMA.

It is thus a feature of at least one embodiment of the invention to provide a spacer that may be readily removed in a manner of conventional bone cement and that is compatible with bone cement.

The spacer may include a series of outwardly extending bosses on a portion abutting the acetabulum, the bosses promoting a uniform layer of bone cement when the socket is installed in the acetabulum.

It is thus a feature of at least one embodiment of the invention to ensure a suitable thickness of the bone cement between the spacer and bone.

The bone cement may be the same material as the spacer.

It is thus a feature of at least one embodiment of the invention to simplify the removal of the spacer and bone cement resulting from their similar properties.

The spacer may be free from metallic reinforcement.

It is thus a feature of at least one embodiment of the invention to provide a spacer that may be removed by breaking it into constituent pieces.

The spacer may provide a substantially semi-spherical outer shell.

It is thus a feature of at least one embodiment of the invention to minimize the material of the spacer to accommodate a variety of different replant situations.

The spacer may provide a rim extending around the polished socket, the rim including apertures extending, at least in part, parallel to an axis of symmetry of the spacer.

It is thus a feature of at least one embodiment of the invention to provide elements assisting in the ultimate removal of the spacer, for example, by permitting the application of torsion or the like.

These particular objects and advantages may apply to only some embodiments falling within the claims and thus do not define the scope of the invention.

### BRIEF DESCRIPTION OF THE DRAWINGS

FIG. 1 is a cross-sectional view of the acetabular spacer of the present invention in position in the pelvis;

FIG. 2 is a perspective view of the acetabular spacer of the present invention showing surface spacers for providing bone cement channels;

FIG. 3 is a cross-sectional view along line 3-3 of FIG. 2 showing an interface between the acetabular spacer and the acetabulum when the temporary acetabular spacer is cemented in place.

### DETAILED DESCRIPTION OF THE INVENTION

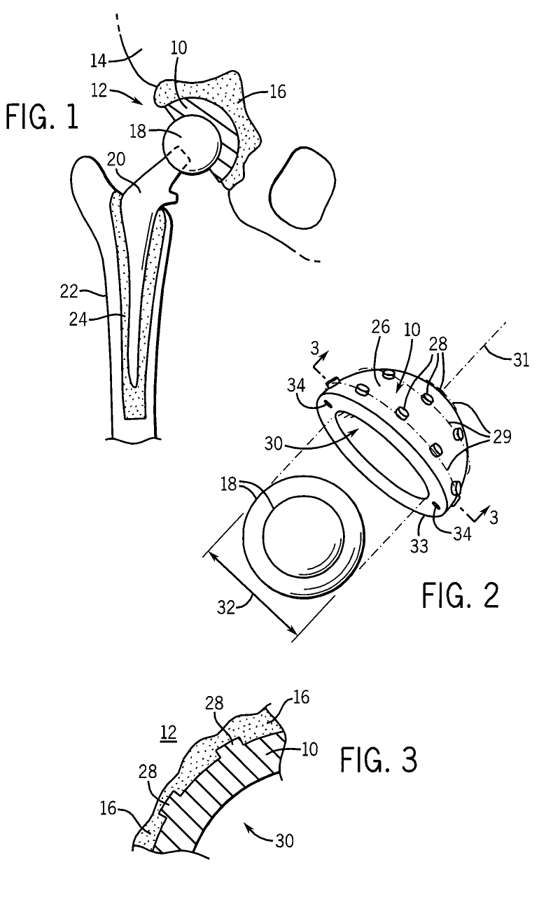

Referring now to FIG. 1, the present invention provides an acetabular spacer 10 that may be held in the acetabulum 12 of the pelvis 14 with bone cement 16. As positioned, the acetabular spacer 10 aligns and spaces a ball head 18 of a femoral prosthesis 20 implanted into the femur 22 as held by additional bone cement 24.

Referring also to FIGS. 2 and 3, the acetabular spacer 10 provides a generally hemispherical shell 26 having an outer surface conforming to the surface of a sphere and having a diameter sized to be received by the acetabulum 12, and having an inner surface also conforming to the surface of smaller sphere to receive the ball head 18. The hemispherical shell 26 may terminate at a lip 33 extending along a plane generally cutting the hemispherical shell 26 along an equatorial plane with respect to the sphere to which the acetabular spacer 10 conforms. The outer surface of the hemispherical shell 26 may be studded with radially extending bosses 28 that space the outer surface of the hemispherical shell 26 from the acetabulum 12 to allow intervening space for the bone cement 16 while allowing the acetabular spacer 10 to be supported against bone of the acetabulum 12.

In one embodiment, the bosses may be cylindrical having a diameter of approximately 3 mm and a height of 0.75 mm and extending along lines of radius of the sphere defining the outer surface of the hemispherical shell 26. It will be understood that the hemispherical shell 26 need not be exactly one half of the sphere but may be semi-spherical conforming to a portion of the sphere somewhat greater than or less than one half of that sphere. In one embodiment, the bosses 28 may be arranged along circles of latitude 29 separated by latitudinal angles of 25° about an axis 31 of radial symmetry of the hemispherical shell 26. In one embodiment, twenty-five bosses 28 may be arranged separated over the outer surface of the hemispherical shell 26. Those bosses 28 closest to a lip 33 may be displaced latitudinally away from the lip 33 to permit an unbroken line of cement 16 around the hemispherical shell 26 at the lip 33. A wall thickness of the hemispherical shell may be approximately 3 mm in one embodiment.

As noted, the acetabular spacer 10 provides a concentric hemispherical bore 30 which may in one embodiment have an inner diameter 32 in the range of 40 to 55 millimeters to receive ball head 18 of femoral prosthesis 20 with a wide range of diameters while still providing for necessary alignment and retention. The concentric hemispherical bore 30 may be polished to reduce wear when in contact with a femur ball. An exposed face of the acetabular spacer 10 may include apertures 34 for receiving and locating insertion tools. For example the apertures 34 may provide receptacles for a wrench providing torque about axis 31 in the manner of the spanner wrench. In one embodiment the apertures 34 are cylindrical borers positioned in opposition at 180° around the lip 33 and extending parallel to the axis.

In one embodiment different standard acetabular spacers 10 may be constructed with the following inner and outer diameters, the latter measured without the bosses 28:

1. Inner diameter: 42 mm; outer diameter 48 mm;

2. Inner diameter: 46 mm; outer diameter 52 mm;

3. Inner diameter: 50 mm; outer diameter 56 mm;

Preferably, the acetabular spacer 10 is constructed of polymethyl-methacrylate (PMMA) infused with a broad-spectrum antibiotic of a type known in the art, for example Gentamicin. Such a material is widely used as bone cement, for example, under the trade name Palacos® as commercially available from Zimmer Inc. having offices in Warsaw, Ind. USA. In this regard, the acetabular spacer 10 may be wholly compatible with the bone cement 16 and may be removed using techniques typically employed for the removal of bone cement 16 taking advantage of its frangible and or meltable qualities.

In practice, a pre-existing acetabular prosthetic socket, for example, constructed of metal or high-density polypropylene, ceramic or the like may be removed from the infected hipbone with appropriate removal of the cement holding the prostheses in place. The acetabular spacer 10 may then be inserted into the open cavity previously holding the prostheses as held by bone cement 16. After a period of healing, the acetabular spacer 10 may be removed, a process facilitated by the ability to melt or break the acetabular spacer 10 into fragments. A new, permanent acetabular prostheses, again of metal, high-density plastic ceramic or the like, is then installed.

It is specifically intended that the present invention not be limited to the embodiments and illustrations contained herein and the claims should be understood to include modified forms of those embodiments including portions of the embodiments and combinations of elements of different embodiments as come within the scope of the following claims.

I claim:

1. A method of treating infection of an acetabular prosthesis comprising the steps of:
(a) removing an existing acetabular prosthesis from an acetabulum;
(b) installing a spacer in the acetabulum formed of a preformed frangible bone cement, cemented with bone cement into the acetabulum, the spacer providing a substantially semi-spherical polished socket for receiving a ball of a femur prosthesis, the spacer having substantially a dimension of the removed acetabular prosthesis; and
(c) after a period of healing, removing the spacer and installing a new acetabular prosthesis.

2. The method of claim 1 wherein the polished socket is no less than 40 mm in diameter.

3. The method of claim 1 wherein the spacer is infused with antibiotic.

4. The method of claim 1 wherein the spacer is constructed of bone cement.

5. The method of claim 1 wherein the spacer is constructed of PMMA.

6. The method of claim 1 wherein the spacer includes a series of outwardly extending bosses on a portion abutting the acetabulum promoting a uniform layer of bone cement when the socket is installed in the acetabulum.

7. The method of claim 1 wherein the bone cement is the same material at the spacer.

8. The method of claim 1 wherein the spacer is free from metallic reinforcement.

9. The method of claim 1 wherein the spacer provides a substantially semi-spherical outer shell.

10. The method of claim 1 wherein the spacer provides a rim extending around the polished socket, the rim including apertures extending, at least in part, parallel to an axis of symmetry of the spacer.

11. An acetabular spacer comprising:
a substantially semispherical shell having an outer surfaces sized for receipt into a acetabulum of a hip and a having an inner semi-spherical socket with a polished surface for receipt of a ball of a femur;
wherein the spacer is constructed of bone cement without metallic insertions and is infused with antibiotic; and
wherein the spacer includes a series of separated bosses extending outward from the outer surface to space the outer surface from the acetabulum thereby promoting a uniform layer of bone cement when the socket is installed in the acetabulum.
